SEQUENCE LISTING (12 sequences)

SEQ ID NO: 1 - DNA, 23 bp, Homo sapiens
tgagagcaatgagcattccgatg

SEQ ID NO: 2 - DNA, 22 bp, Homo sapiens
cagggagtttccatgaagccac

SEQ ID NO: 3 - DNA, 22 bp, Homo sapiens
aacaaccccacaccccagtttg

SEQ ID NO: 4 - DNA, 20 bp, Homo sapiens
ttgaagcaggcatcagtcac

SEQ ID NO: 5 - DNA, 22 bp, Homo sapiens
tggtgctgttttctacccttgg

SEQ ID NO: 6 - DNA, 24 bp, Homo sapiens
atctctgcctctgtcttcacacgg

SEQ ID NO: 7 - DNA, 22 bp, Mus musculus
cgcaccctatgaggacaaaaag

SEQ ID NO: 8 - DNA, 23 bp, Mus musculus
ggaatgccagattacaaacgctg

SEQ ID NO: 9 - DNA, 22 bp, Homo sapiens
gtgggaacgcactaacttgacc

SEQ ID NO: 10 - DNA, 23 bp, Homo sapiens
cctggtgaagatgagaggtgatg

SEQ ID NO: 11 - DNA, 24 bp, Homo sapiens
gcagaatgagtgctcctaaactcc

SEQ ID NO: 12 - DNA, 20 bp, Homo sapiens
cctgatgatgagccacagcg

(19) United States
(12) Patent Application Publication
(10) Pub. No.: US 2005/0143334 A1
(43) Pub. Date: Jun. 30, 2005

(54) Genetic markers and methods for the diagnosis, treatment and prevention of tumor metastasis

(76) Inventors: David Tarin, San Diego, CA (US); Steven Goodison, Jacksonville, FL (US); Virginia Benedicta Urquidi, Carlsbad, CA (US)

Correspondence Address: GORDON & REES LLP, 101 WEST BROADWAY, SUITE 1600, SAN DIEGO, CA, 92101, US

(21) Appl. No.: 10/881,822

(22) Filed: Jun. 22, 2004

(30) Foreign Application Priority Data
Dec. 20, 2002 (WO) PCT/US02/41195

(51) Int. Cl. A61K 48/00; C12Q 1/68

(52) U.S. Cl. 514/44; 435/6

(57) ABSTRACT

The multi-step nature of metastasis poses difficulties in both design and interpretation of experiments to unveil the mechanisms causing the process. In order to facilitate such studies a pair of breast tumor cell lines that originate from the same breast tumor, but have diametrically opposite metastatic capabilities have been derived. Comparison of the two cell lines has revealed a number of genes that are differentiallay expressed. The invention is the identification of these differences. The invention is the use of knowledge of the differential gene expression to develop novel therapeutic and diagnostic methods for cancer.

### CROSS-REFERENCES TO RELATED APPLICATIONS

This application claims the benefit of priority of U.S. provisional application Ser. No. 60/342,298 filed Dec. 20, 2001 which is incorporated herein by reference in its entirety.

### BACKGROUND OF THE INVENTION

Despite significant advances in the treatment of primary cancer, the ability to predict the metastatic behavior of a patient's cancer, as well as to detect and eradicate such recurrences, remains the greatest clinical challenge in oncology. To make progress in this area it is essential to obtain more detailed knowledge of the molecular mechanisms involved in metastasis as a basis for novel approaches to the evaluation of individual patient prognosis and to rational therapeutic design.

The formation of a secondary tumor colony in a distal site is the culmination of a complicated series of sequential and highly selective events. To succeed in accomplishing lymphogenous or hematogenous metastasis, tumor cells must have invaded the local extracellular matrix and penetrated the vascular endothelium to gain access to the circulation for transport away from the primary site. In the next phase of the process, cells that survive the physical stresses of the circulation and surveillance by body defense mechanisms lodge into the capillary bed of a conducive tissue or organ and exit through the vessel wall. Finally, to thrive and form secondary deposits, the tumor cells must proliferate and attract a new vascular supply and other supporting cells from the host tissue. These consecutive events are dependent upon the coordinated regulation of gene expression.

This multistep nature of metastasis poses difficulties in both design and interpretation of experiments to unveil the mechanisms causing the process. Studies on excised fixed human tissues are complicated by the variance of genetic background between individuals and by the cellular heterogeneity of a complex tissue mass. Breast cancer is also a collection of distinct diseases, and it can be difficult to be certain of the histogenetic classification of the tumor in advanced cases, thereby causing an inappropriate combination of data from genetically distinct lesions. However, the major limitation of such studies is the inability to identify those cells in a tumor mass that are truly capable of metastasis. Even if derived from a single cell, an advanced carcinoma is a mixture of genotypically and phenotypically distinct cells, and only a tiny fraction of those cells may possess the ability to disseminate from the primary lesion. Furthermore, it is estimated that only 0.1% of tumor cells that do enter the circulation will form secondary deposits in a distal organ. Analysis of actual metastases may not be helpful either, because these cells have proliferated in a non-breast tissue environment and therefore may display a markedly different molecular profile and may not retain the ability to metastasize again.

Critical to the experimental analysis of metastasis has been the isolation of human tumor cell lines and the ability to study their behavior in vivo by inoculation into immune-compromised mice. Several established human breast cancer cell lines with varying documented abilities of invasiveness and/or migration in vitro are available, and some are capable of spontaneous metastasis in vivo, i.e., dissemination from growth in the mammary gland and proliferation in a distal site. However, most of these are polyclonal and composed of cell populations that are heterogeneous in metastatic phenotype, making them difficult to use as models in studies seeking to define genes causing metastasis. Several laboratories have obtained cell lines with increasing metastatic phenotype by recovering and culturing metastatic deposits derived from primary inoculations and recycling the cells through several rounds of orthotopic selection. The resulting cell lines represent improved models for studying metastasis because they are mono- or oligoclonal and, therefore, of uniform phenotype. The most common difficulty with these models is the lack of a corresponding totally nonmetastatic, clonally uniform counterpart for comparison, because the selection process used for the derivation of metastatic lines cannot be used for the selection of the converse phenotype. The original cell line, from which the hypermetastatic cell line is selected, is not an appropriate counterpart for comparison because it is a heterogeneous polyclonal resource containing many clones of differing metastatic propensity.

Despite decades of research, no single consistent marker or effector of metastatic behavior has yet been identified. Given the complexity of the metastatic process during which a disseminating tumor cell must accomplish several sequential tasks and survive in many different environments, it is far more likely to be a set of genes that is responsible for manifesting the overall phenotype. Modern technological advances now permit the application of high throughput gene expression analysis, by methods such as gene-chips and spotted microarrays, to identify whether coordinated patterns of expression of clusters of genes are involved in this complex process. The performance of such analysis is limited by a lack of an appropriate experimental system.

Relatively few studies have produced functional data demonstrating the role of candidate genes in the regulation of human breast tumor cell metastasis. A number of metastasis-suppressorgenes have been proposed, including KAI1, Nm-23-H1, BRMS-1, KISS-1 and TSP-1, molecules that have been reported to suppress metastasis in breast cell lines and in other experimental systems. Recently, a novel gene was implicated as a breast metastasis supressor gene. Based upon cytogenetic data that identified chromosome 11 as containing multiple genetic aberrations associated with breast cancer progression, Phillips et al. (1996) showed that introduction of a normal chromosome 11 into MDA-MB-435 cells significantly reduced the metastatic potential of the cell line without affecting tumorigenicity. Subsequent analyses identified the location of one of the genes responsible for this effect, and it was named breast metastasis suppressor-1 (BRMS-1) (Seraj, 2000). Stable transfection of BRMS-1 cDNA into MDA-MB435 and MDA-MB-231 cell lines correlated with locally invasive tumor growth in athymic mice and with significant reduction of metastases to the lungs and lymph nodes.

A pair of breast cell lines have been recently developed to facilitate the identification of factors that promote or inhibit metastasis. The two monoclonal cell lines were isolated by limiting dilution from the polyclonal MDA-MB-435 breast cancer cell line. When inoculated into the mammary fat pad of athymic mice, both cell lines form primary tumors but, whereas clone M-4A4 aggressively metastasizes to the lung and lymph nodes, the NM-2C5 clone is entirely non-metastatic. This experimental system of analysis enables comparative molecular screening and functional evaluation of candidate metastasis-related genes in an isogenic background (Urquidi V et aL, 2002). Expression may be assayed by any of a number of methods including, but not limited to, subtractive hybridzation, ELISA, quantitative polymerase chain reaction (PCR), gene chip, western blot and northern analysis.

### SUMMARY OF THE INVENTION

The invention involves the discovery of the differential regulation of a number of genes in the two cell lines, the metastatic M-4A4 cell line and non-metastatic, but equally tumorogenic NM-2C5 cell line. Matrix-metalloprotienases-8 and -17, tyrosinase-related protein-1, cytokeratin-9 and thrombospondin-1 were all found to be up-regulated in the non-metastatic cell line NM-2C5. Collagen IX α-1, CD27-ligand (TNFSF7) and osteopontin-1 were all found to be upregulated in the metastatic cell line M-4A4. Identification of metastasis promoting and inhibiting factors provides a new paradigm for cancer therapy, diagnosis and prognosis.

The invention also involves a method to prevent metastasis or treat metastatic disease by the identification of genes differentially expressed in the process. Genes or gene products that are expressed in non-metastatic cells can be used as therapeutics or the basis for the development of drugs to inhibit the metastasis of tumors. Such agents can be used in conjunction with other cancer therapies (e.g. chemotherapy, surgery, radiation) to prevent or inhibit the growth of metastasis. Alternatively, compounds can be developed to block the gene expression (e.g. antisense oligonucleotides) or the activity of the gene products associated with metastasis. Dominant negative forms of metastasis promoting factors can be administer to inhibit metastasis of tumors.

The invention further involves the use of the expression of metastatic and non-metastatic markers in the diagnosis, staging and prognosis of cancer. Analysis of expression of various metastatic and non-metastatic markers can be used to aid in the staging of a tumor. Such markers can be used to determine the metastatic potential of a tumor, providing a prognostic indicator of disease. A tumor expressing metastatic markers needs to be treated more promptly and aggressively with more whole body therapies (e.g. chemotherapy) rather than with more localized therapies (e.g. surgery, radiation). Thus, a patient with a non-metastatic tumor may be spared from undergoing chemotherapy.

As many of the metastasis promoting proteins are secreted and present in bodily fluids, expression can be assayed in any of a number of fluids including blood, lymph or other bodily fluid using an immune assay such as an ELISA. Such a non-invasive method is ideal for the monitoring of disease progression or regression. Expression of markers does not need to be performed on the tumor itself. However, early stage tumors ortissue collected during biopsy can be assayed using any of a number of protein or nucleic acid based assays including quantitative real time PCR (RT-PCT). Expression can be assayed by a number of other methods well known to those skilled in the art.

The invention still further involves a method to develop novel therapeutics and interventions in cancer. Therapeutics can be based on the structure of gene products found to be expressed in the non-metastatic phenotype. Alternatively, the coding sequences for such genes can be delivered by any of a number of gene transfer methods for the treatment of disease. Therapies related to dominant negative forms of metastasis promoting genes and gene products can be developed. Alternatively, methods to inactivate or cause the mislocalization or processing of the metastasis promoting gene products can be developed as therapeutics.

### DETAILED DESCRIPTION OF THE PREFERRED EMBODIMENTS

The characterization of the M4A4 and NM-2C5 paired cell line model is presented, comparing their molecular profiling (see Table 1, page 7 below) and in vitro-and in vivo growth rates (see Table 2, page 13 below). The cell lines have been demonstrated to originate from the same genetic source by spectral karyotyping. Data are presented on the expression of a number of gene products previously implicated in transformation and metastasis by RNA and immunochemical analyses. The sequences of these genes and gene products are known and may be readily obtained using a searchable database such as BLAST or GenBank using methods well known to those skilled in the art. The majority of such targeted comparative analyses revealed equal levels of gene expression in both clonal cell lines, further supporting the tightly controlled nature of the experimental system.

Major differences were revealed in a small number of genes and gene products by multiple analyses, as is evident from Table 1 on the following page, which presents comparative gene expression analysis of NM-2C5 and M-4A4 human breast cells. The expression of matrix-metalloproteinase-8 and 17 (MMP-8 and -17), tyrosinase related protein-1 (TYRP-1), cytokeratin-9 and thrombospondin-1 (TSP-1) were found to correlate with the nonmetastatic phenotype. The expression of collagen IX, α-1 (Coll IX α-1), CD27 ligand (TNFSF7) and osteopontin (OPN) and to a lesser extent, the expression of tissue inhibitor of matrix metalloprotienase-1 (TIMP-1) were found to correlate with a metastatic phenotype. Verification of the differential expression of these genes in cultured cells and in xenograft tissues was achieved using quantitative RNA and protein analyses.

TABLE 1
Comparative gene expression analyses of NM-2C5 and
M-4-A4 human breast cells^{a}

| | Presence (P) or | Analytical | Expression |
|---|---|---|---|
| Gene transcript/protein | absence (A) | method | increased in: |
| ER-α | A/A | I, W, R | |
| ER-β | P/P | R | equal |
| Progestrone receptor | A/A | I | |
| EGFR^{b} | P/P | I | equal |
| Ki67 | P/P | I | equal |
| Cytokeratin | A/A | I | |
| Cytokeratin-9 | P/P | | NM-2C5 |
| Collagen IX α-1 | P/P | | M-4A4 |
| EBV | A/A | I | |
| BCL-2 | P/P | I | equal |
| CD27-ligand (TNFSF7) | P/P | | M-4A4 |
| CEA | A/A | I | |
| Maspin | P/P | W | equal |
| Paxillin | P/P | W | equal |
| PTEN | P/P | W | equal |
| NG2 | P/P | W | equal |
| TSP-1 | P/P | I, W, R | NM-2C5 |
| CD44 | P/P | I, W, R | equal |
| Nm23-H1 | P/P | W | equal |
| KiSS-1 | P/P | R | equal |
| KAI-I | A/A | R | |
| E-cadherin | A/A | R | |
| RhoC | P/P | R | equal |
| BRMS-1 | P/P | R | equal |
| OPN | P/P | I, W, R | M-4A4 |
| c-myc | P/P | R & W | equal |
| Ras | P/P | I | equal |
| P53 | P/P | I | equal |
| MMP2 | P/P | W | equal |
| MMP8 | P/P | W, R, D | NM-2C5 |
| MMP9 | P/P | W | equal |
| MMP17 | P/P | | NM-2C5 |
| TIMP2 | P/P | W | equal |
| TIMP-1 | P/P | W | M-4A4 |
| TYRP-1 | P/P | W, R, D | NM-2C5 |

^{a}Cultured tumor cells were analyzed for transcript and/or protein expression by Western blotting (W), Immunohistochemistry (I), RNA analyses (R) using Northern blot and/or PCR amplification, or representational difference analysis (D).
^{b}EGFR, epidermal growth factor receptor;
CEA, carcinoembryonic antigen.

The knowledge of specific genes and gene products that are upregulated in non-metastatic tumors provides a new approach for chemotherapy to prevent the metastasis of tumor allowing for localized treatment with methods such as surgery or radiation. Genes and gene products upregulated in non-metastatic tumors and agents designed to act in a similar manner are grouped into a class called non-metastatic-agents. This includes the specific genes and gene products identified in the instant application as well as any other natural or synthetic agents developed based on the specific genes or gene products identified herein. Additionally, non-metastatic factors would include agents designed to upregulate the expression or activity of the genes or gene products identified herein.

Proteins are known to have the ability to suppress tumor formation throughout the body. For example, thrombospondin-1 has been implicated in the phenomenon of concomitant tumor resistance. This refers to the ability of some large primary tumors to hold smaller, distant tumors in check, preventing their progressive growth. Therapy based on administration of non-metastatic agents would likely be coupled with other interventions for treatment of the primary tumor. However, agents to inhibit metastasis can be used alone with inoperable tumors or those refractile to other chemo-therapeutic agents to restrict the disease to a single location within the body.

Therapy based on the non-metastatic agents can include the delivery of a gene or gene fragment to the patient in a nucleic acid expression cassette. A variety of gene therapy and gene delivery protocols are well known to those skilled in the art. Protocols vary depending on the material to be delivered and the duration of the therapy. The exact method of delivery of the therapeutic gene or gene product is not a limitation of the invention. As such proteins are typically secreted, the exact location of gene delivery and expression is less limiting than in many other gene therapy protocols. Pharmacological agents to modify expression of genes expressed by non-metastatic tumors can also be used to the same effect.

Natural, synthetic or modified proteins or portions thereof, expressed by non-metastatic tumors can be delivered as therapeutic agents and are non-metastatic agents. Structural data on proteins expressed by non-metastatic tumors can be used as a basis for rational drug design to develop peptidomemetics and other small molecule pharmacological agents to act in a manner similar to the protein in inhibiting metastasis.

In the case of MMP-8 and -17 and other proteins that must be activated, agents could be administered to increase the activation of the proteins increasing the effective concentration of the protein in the patient are also non-metastatic agents.

The identification of metastasis promoting factors allows for the development of agents to inhibit the production or action of the metastasis promoting factors. Such agents are called metastasis inhibiting agents and can include a number of classes of molecules. Delivery of antisense oligonucleotides can inhibit the expression of the metastasis promoting factors. Oligonucleotides may be delivered using an expression cassette in a manner similar to those use in gene therapy. Alternatively oligonucleotides, preferably modified for increased stability (e.g. phosphorthioate backbone, L-nucleic acids) can be used. Pharmacological agents may be given to decrease expression of the metastasis promoting factors. The activity of metastasis promoting factors can be decreased by the delivery of dominant negative forms of the factors either by direct protein delivery or by the use of any of a number of gene delivery protocols. The method of delivery of the agent and the exact composition are not limitations of the instant invention.

Gene products that promote metastasis can be used as the basis for rational drug design by determining essential regions of the metastasis promoting gene product and developing agents to block the active sites on the protein. Alternatively, antagonists that bind the receptors as the metastasis promoting factors can be developed to both inhibit binding and downregulate the activity of the receptor.

The identification of genes and gene products that are associated with metastatic or non-metastatic disease, collectively called metastasis regulating genes and gene products, can be used as both diagnostic and prognostic indicators of disease. A test to predict the likelihood of metastasis can both reduce the cost of medical care and increase the quality of life for one diagnosed with cancer. Slow growing tumors with low potential for metastasis can be treated with “watchful waiting” as the chances of the tumor growing sufficiently to result in other pathology are minimal. A small tumor with high metastatic potential can be treated aggressively with surgery at an earlier stage than such an invasive method would normally be used.

Any of a number of diagnostic tests could be used based on nucleic acid or protein expression. ELISA assays can be used as many of both the promoting and inhibiting factors are secreted proteins and present in circulation. Such a non-invasive method is ideal for the monitoring of the levels of factors in the circulation. Alternatively, analysis can be performed by any of a number of nucleic acid based tests including PCR based assays which can be performed using a minimal amount of sample (e.g. portion of a needle biopsy), as well as northern and Southern blots. Such methods are well known to those skilled in the art. Methods for analysis of gene and protein expression and activity in cell lines and mouse tumors and tissues are detailed in the Examples below. The methods can be readily adapted for analysis of patient samples. Methods for obtaining patient samples and isolation of nucleic acids and proteins from such samples are well known to those skilled in the art. The selection of the method for performing the test is not a limitation of the invention.

### EXAMPLE 1

Isolation of Clonal Cell Lines and Culture Conditions. Clonal sublines of the metastatic, polyclonal MDA-MB435 human breast carcinoma line (10, 13) were isolated by the limiting dilution technique, with direct microscopic monitoring of monocellular origin. Monoclonal cell lines were propagated in RPMI 1640 supplemented with 10% newborn calf serum (Invitrogen, Carlsbad, Calif.) at 37° C. in a humidified atmosphere of 5% CO₂-95% air. Cells were harvested by washing the monolayer with PBS and briefly incubating with 0.25% trypsin/0.02% EDTA. The detached cells were washed by centrifugation and resuspended in RPMI 1640 media and counted before passaging or inoculation. Two selected clones of nonmetastatic and metastatic phenotypes (NM-2C5 and M-4A4, respectively) were propagated for further investigation. Analyses were performed on cultures passaged no more than 10 times from frozen stock vials designated passage 1 at the time of in vivo inoculation. Cultures were tested and declared free of Mycoplasma and common murine pathogens.

### EXAMPLE 2

Tumorgenicity and Metastasis Formation in Vivo. Female athymic mice (MF1Nu strain) were housed in an isolation suite for the duration of the experiments. The tumorigenicity and spontaneous metastatic capability of the cell lines were determined by injection into the mammary fat pad. One million cells in 0.05 ml of a 1:1 mixture of RPMI 1640 medium and ECM gel (Sigma Chemical Co., St. Louis, Mo.) were inoculated into the anesthetized mouse. Animals were monitored every 2 days for up to 5 months for tumor growth and general health. The rate of primary tumor growth of the clones was determined by plotting the means of two orthogonal diameters of the tumors, measured at 7-day intervals. Animals were sacrificed and autopsied at 3-5 months postinoculation, unless moribund earlier. Metastasis formation was assessed by macroscopic observation of all major organs for secondary tumors and confirmed by histological examination of organs and lymph nodes. Tissue samples harvested for histological analysis were either fixed and embedded in paraffin wax or snap-frozen in liquid nitrogen.

Clonal cell line M-4A4 was found to be more aggressively metastatic to the lungs than the parent cell line and to spread only to the lungs and lymph nodes. Rapidly formed primary tumors (palpable within 2-3 weeks) were evident in all mice examined. Fifteen of 19 mice (79%) had metastases at either secondary site, with 74% of mice having detectable lung metastases. Only one mouse had lymph node metastases in the absence of lung metastases. Conversely, clonal cell line NM-2C5 was shown to be completely nonmetastatic from the orthotopic inoculation site. NM-2C5 primary tumors proliferated at a slower rate than M-4A4 in vivo (median 153 and 98 days, respectively, to the end point of a single orthogonal 2-cm diameter). However, on examination of 17 mice, no metastases were found in any organ, although the primary tumors were in situ up to 44 days longer. This may be seen in Table 2 on the following page, which presents the data on tumorgenicity and production of metastasis by human breast cancer cells orthotopically implanted in athymic mice. No mice appeared to have any ill-effects from M-4A4 or NM-2C5 tumor growth within the time frame of the experiment. These two carefully scrutinized clones of opposing metastatic phenotype were therefore selected for subsequent analysis of differential gene expression. These particular clones appear to be remarkably stable with respect to the metastatic phenotype. Cells are cultured in vitro anywhere up to passage 10 before inoculation into the athymic mice, but due to the length of incubation of the orthotopically implanted primary tumor before sacrifice (3-5 months; Table 2). the cells must go through many tens of passages in vivo. However, this extended passage in vivo does not result in phenotypic drift with regard to metastatic potential, suggesting a high level of genomic stability.

TABLE 2
Tumorigenicity and production of metastasis by human breast cancer
cells orthotopically implanted in athymic mice

| | | Median days | Median size of | No. of Animals | | Median lung |
|---|---|---|---|---|---|---|
| | No. of | postinoculation | primary tumor | with Metastasis | | metastatic |
| Cell line | Mice | (Range) | (cm³) (Range) | Lymph nodes | Lungs | deposits (Range) |
| MDA-MB- | 17 | 98 (63-130) | 3.8 (0.56-7.28) | 12/17 (71%) | 9/17 | 6 (3-20) |
| M-4A4 | 19 | 98 (56-112) | 6 (2.46-8) | 8/19 (42%) | 14/19 | 7 (1-50) |
| NM-2C5 | 17 | 153 (73-174) | 4.28 (0.33-7.83) | 0/17 | 0/17 | 0 |

### EXAMPLE 3

Differential Expression of TSP-1 and OPN. Increased TSP-1 expression in NM-2C5, relative to its metastatic counterpart M-4A4 was first demonstrated using comparativetotal protein analysis of the conditioned media (CM) recovered from the cultured cell lines. Analysis of secreted proteins revealed the presence of a 150-kDa polypeptide at high concentration in NM-2C5 supernatants, which was virtually absent in supernatants of M-4A4 cells grown under identical conditions. Tryptic digestion and mass spectrometry of the protein excised from the gel identified the differentially expressed protein as TSP-1. This identity was verified with specific TSP-1 antibodies using Western analysis. The differential expression of the TSP-1 gene between the two cell lines was also confirmed at the transcriptional level by measuring the relative abundance of steady-state TSP-1 mRNA on Northern blots and revealed a differential expression of approximately 15-fold.

OPN, a secreted calcium-binding phosphoprotein, was targeted specifically for differential expression analysis. Although poorly understood functionally, OPN has recently been linked to tumorigenesis and metastasis in experimental animal models and human patient studies. Initial Northern blot analysis of OPN transcript expression in the paired cell line model revealed approximately 30-fold more OPN in M-4A4 cells relative to NM-2C5. The differential expression of secreted OPN protein was verified by immunoblotting of CM.

Having determined the differential expression of TSP-1 and OPN in M-4A4 and NM-2C5 cultured cells, the expression of TSP-1 and OPN transcripts was examined in primary xenograft tissue recovered from nude mice. Real-time quantitative PCR analysis of three primary tumors originating from each clone revealed that OPN and TSP-1 transcripts were also differentially expressed in vivo. Real-time quantitative PCR results (normalized against average values for the housekeeping gene GAPDH) revealed thatTSP-1 mRNA was 22-fold higher in NM-2C5 tissue relative to M-4A4, and OPN mRNA was 21-fold higher in M-4A4 tissue relative to NM-2C5. Real-time PCR evaluation of TSP-1 and OPN expression in cultured cells revealed 33-fold and 88-fold differential expression, respectively (lower fold differences revealed using Northern and Western analyses were presumably underestimated by solid-phase filter analyses). Immunochemical analysis of xenograft tissue revealed that both OPN and TSP-1 were expressed, primarily accumulating in the extracellular matrix, but truly quantitative protein analysis was unattainable because both are secreted proteins.

Cytogenetic evaluation of the cell lines was used to estimate gene dosage and to assess the gross integrity of the chromosomal structure at the OPN and TSP-1 loci. OPN is a single-copy gene located on chromosome 4, and both NM-2C5 and M-4A4 cell lines were found to contain two copies of this chromosome of normal constitution. TSP-1 is also a single-copy gene, located on chromosome 15 at position 15q15. Chromosome 15 does show some rearrangement in our cell lines, but this does not appear to affect the TSP-1 gene. Two chromosomes 15′ with structural integrity at 15q15 were always present in both cell lines. Furthermore, the chromosome 15 translocation prevalent in NM-2C5 cells has a breakpoint mapped as t(12;15)(q22;q26.1), and the t(8;15) translocation common to both cell lines is a t(8;15)(q24;q21), as determined by G-banding and FISH.

### EXAMPLE 4

MMP-8 and TYRP-1 mRNAs are overexpressed in the non-metastatic NM-2C5 cell line relative to the metastatic M-4A4 cDNA-RDA (Representational Difference Analysis) is a process of subtractive hybridization coupled to PCR amplification used to identify genes differentially expressed between two different cell populations. A modified protocol of Hubank and Schatz (Hubank and Schatz, 1 994) was used to generate a subtracted cDNA library for the NM-2C5 cell line. An initial screening of an array of 90 library clones with cDNA from both cell lines was conducted to select those genes with the highest difference in levels of expression. Twenty cDNA-RDA clones derived from the NM-2C5 subtracted library were thus selected-and used as-probes in Northern analysis for hybridization with mRNA from both NM-2C5 and M-4A4 cell lines. Clones hybridizing to transcripts of ˜3.3 kb and ˜2.7 kb, found to be the most differently expressed, were identified as fragments of MMP-8 and TYRP-1 respectively, by sequence analysis. This difference in levels of expression in vitro was maintained in the tumors formed by NM-2C5 and M-4A4 cells after orthotopic implantation in athymic mice.

Comparison of the northern signals for MMP-8 and TYRP-1 mRNAs relative to those of P-actin performed by phosphorimaging analysis, revealed a ˜20-fold difference in expression of the 2 genes between NM-2C5 and M-4A4, in vitro and in vivo. In order to rule out the possibility of a contribution of MMP-8 mRNA from host tissues when using tumor-derived mRNA on a northern blot, RT-PCR was performed with primers designed to specifically amplify either the mouse or the human 3′ untranslated region of the MMP-8 mRNA after reverse transcription. Amplification results indicated that RT-PCR failed to detect mouse MMP-8 transcripts in the tumor samples, and that the northern hybridization signals represented only human MMP-8 mRNA.

### EXAMPLE 5

MMP-8 and TYRP-1 proteins are over-expressed in the non-metastatic NM-2C5 cells. The differential expression pattern of MMP-8 and TYRP-1 mRNA between the two cell types was also observed at the protein level. To examine MMP-8 protein expression and secretion, concentrated conditioned medium and lysates from NM-2C5 and M-4A4 cells, and extracts derived from the respective primary tumors were analyzed by SDS-PAGE under reducing conditions and western blotting. A monoclonal antibody directed against human recombinant MMP-8 detected a 65 kDa immunoreactive protein over-expressed in conditioned medium from NM-2C5 cells. The Mr of glycosylated MMP-8 synthesized by neutrophils is reported as 70 and 89 kDa-corresponding to active and latent forms of the enzyme respectively whereas MMP-8 from gingival crevicular fluid migrates at 78 and 60 kDa suggesting that the 65 kDa form of MMP-8 detected has been activated by proteolytic cleavage of the prodomain or represents the proenzyme with a lesser degree of glycosylation. MMP-8 protein in the NM-2C5 or M-4A4 cell lysates or in the primary tumors could not be detected by western analysis, indicating that the protein does not accumulate intracellularly. The expression of the membrane protein TYRP-1 was readily detected in the NM-2C5 cells both in vitro and in the primary tumors, whereas it could not be detected in M-4A4 cells or the tumors that they formed.

### EXAMPLE 6

MMP-8 secreted from non-metastatic NM-2C5 cells displays proteolytic activity following activation. To investigate whether the non-metastatic NM-2C5 cells secrete enzymatically functional MMP-8, conditioned media from NM-2C5 and M-4A4 cells was analyzed by non-reducing SDS-PAGE followed by zymography. A protein with caseinolytic and gelatinolytic activity was detected in the NM-2C5 but not in the M-4A4 conditioned medium. The relative mobility (Mr) of this protein was identical to that of an immunoreactive protein detected, with an MMP-8 monoclonal antibody, in NM-2C5 conditioned medium, but not in that conditioned by M-4A4. Pro-MMP exposure to SDS leads to activation without pro-peptide cleavage and thus recombinant pro-MMP-8 also displays proteolytic activity in this assay. These results indicate that the NM-2C5 cells are producing and secreting an activatable MMP-8 that is capable of proteolysis.

### EXAMPLE 7

Evaluation of MMP-2, MMP-9, TIMP-1 and TIMP-2 expression patterns in NM-2C5 and M-4A4 cells. In view of the evidence for a role of the gelatinases MMP-2 and MMP-9 and their inhibitors in metastasis, their relative expression levels were investigated in the paired breast metastasis model by western analysis. Similar expression levels of MMP-2, MMP-9 and TIMP-2 were observed in M-4A4 and NM-2C5 cells. However, TIMP-1 expression in the metastatic M-4A4 cells is increased approximately 3-fold in comparison to its expression in NM-2C5 cells. These results indicate that the lack of metastatic potential of NM-2C5 cells cannot be explained by the increased expression of either TIMP-1 or TIMP-2. Conversely, the metastatic behavior of M-4A4 does not appear to be due to an increase in expression of either MMP-2 or MMP-9 by these cells.

### EXAMPLE 8

Down-regulation of MMP-8 increases the invasiveness of NM-2C5 cells in vitro. In order to evaluate the functional role of MMP-8 and TYRP-1, the M-4A4 cells were stably transduced with either MMP-8, TYRP-1 or both genes. The change in levels of expression of these proteins between the M-4A4 original cell line and its derivatives (M-4A4-M8, M-4A4-TYRP-1; M-4A4-Neo and M-4A4-Hygro) was analyzed. M-4A4-M8 cells express MMP-8 at levels much greater than NM-2C5 cells, as found by immunoblotting and gel zymography. Similarly, the expression of TYRP-1 in M-4A4-TYRP-1 is considerably higher than that of NM-2C5 cells. The expression of MMP-8 in NM-2C5 cells was reduced by stable retroviral transduction with antisense MMP-8 cDNA. The resulting cell line, NM2C5-ASM8, did not express detectable amounts of MMP-8 either by Western analysis or by gelatin zymography. High density oligonucleotide array (Affymetrix GeneChip) analysis of the level of transcription of gelatinases (MMP-2 and MMP-9) and collagenases (MMP-1 and MMP-13) revealed that these metalloproteinases were not affected in NM2C5-ASM8 cells with reference to the original NM-2C5 cells. The migration and the invasive potentials of the original M-4A4 and NM-2C5 cells was assessed using a modified Boyden chamber assay. It was found that although their migration across an uncoated porous membrane (8 μm pores) in response to serum as chemoattractant was not significantly different, M-4A4 cells were significantly more invasive than NM-2C5 cells (P<0.05) across the same membrane coated with Matrigel. Manipulation of the cell lines by altering the expression of MMP8 or TYRP-1 alone or in combination had no effect on their migration. However, down-regulation of MMP-8 expression in NM-2C5 cells had a significant effect on invasion. Knock-down of MMP8 expression by antisense perturbation in 2C5-ASM8 cells resulted in a 2.5-fold increase in invasion over both the NM-2C5, and the vector-alone transduced 2C5-Neo cell lines (P<0.05), and resulted in an even more invasive capacity than the metastatic M-4A4 cells. The over-expression of MMP-8 and/or TYRP-1 had no significant effect on the invasive capacity of M-4A4 cells.

### EXAMPLE 9

RT PCT. Cultured cells were grown to ˜75% confluence before extraction of RNA. Frozen tissues recovered from nude mice were sectioned on a cryostat, and 100 sections (5 μm) were used for RNA extraction. Total RNA was isolated using an RNeasy kit (Qiagen, Valencia, Calif.), and mRNA was purified with Oligotex (Qiagen), treated with DNase I, and reverse transcribed using Moloney murine leukemia virus reverse transcriptase with a combination of oligodeoxythymidylic acid and random decamers (Ambion, Austin, Tex.). The resulting cDNA was used as a template for PCR using gene-specific primers. Primer sequences used were: OPN (GenBank accession no. AF052124), forward primer 5′-TGAGAGCAATGAGCATTCCGATG, reverse primer 5′-CAGGGAGTTTCCATGAAGCCAC; TSP-1 (GenBank accession no. X14787), forward-5′-AACAACCCCACACCCCAGTTTG, reverse 5′-TTGAAGCAGGCATCAGTCAC. PCR primers were designed, based on the human (Genbank NM_{—}002424) and mouse (Genbank NM_{—}008611) MMP8 cDNA sequences (Hasty et al., 1990; Lawson et al., 1998), to specifically amplify either human or mouse MMP-8 mRNA and to avoid amplification of other MMPs. Human MMP-8 sense primer 5′-TGGTGCTGTTTTCTACCCTTGG, antisense primer 5′-ATCTCTGCCTCTGTCTTCACACGG (pair generates a 268 bp amplification product), mouse MMP-8 sense primer 5′-CGCACCCTATGAGGACAAAAAG, antisense primer 5′-GGAATGCCAGATTACAAACGCTG (pair generates a 130 bp amplification product). A pair of sense and antisense primers located in different exons of the human MMP-8 gene was also used in order to rule out contaminating genomic DNA amplification: sense primer 5′-GTGGGAACGCACTAACTTGACC (nt 392-413, exon 2), antisense primer 5′-CCTGGTGAAGATGAGAGGTGATG (nt 499-521, exon 3). The predicted size of cDNA amplification products with this set of primers is 130 bp whereas amplification of genomic DNA would produce a 795 bp fragment. Exon-intron boundaries were determined by alignment of the human MMP-8 cDNA sequence with contigs of the genomic clones with Genbank accession #AP0006472 and AP000922 (working draft sequences). TYRP-1 sense primer; 5′-GCAGAA TGAGTGCTCCTAAACTCC; TYRP-1 antisense primer; 5′-CCTGATGATGAGCCACAGCG (pair generates a 187 bp amplification product). Hot-start PCR conditions were performed, and cycling conditions were adjusted for primer pair characteristics and estimated transcript abundance. Amplification products were resolved by agarose gel electrophoresis. For verification of specificity, products were recovered from gels and sequenced directly using the amplification primers using automated sequencing.

### EXAMPLE 10

Quantitative PCR Analysis. Total RNA was isolated using the RNeasy kit (Qiagen), and DNA was removed by digestion with RNase-free DNase A (Ambion). cDNA was synthesized using Moloney murine leukemia virus reverse transcriptase and an oligodeoxythymidylic acid primer (Ambion). PCR was performed using the SYBR Green PCR Master Mix kit containing SYBR green I dye, AmpliTaq Gold DNA Polymerase, deoxynucleotide triphosphates with dUTP, passive reference, and optimized buffer components (PE Applied Biosystems). PCR primers were designed against the 3′-UTR of the human target genes using MacVector software (Oxford Molecular, Beaverton, Oreg.) and checked for the absence of potential binding to mouse homologue sequences. All primers were used at a final concentration of 100 nM and 1 μl of cDNA dilution was added in 25 μl PCR reactions. No-template controls were included for each target. Thermocycling was initiated with a 10 min, 95° C. enzyme activation step followed by 40 cycles of 95° C. for 15 s and 60° C. for 1 min. All reactions were done in triplicate, and each reaction was gel-verified to contain a single product of the correct size. Data analysis was performed using the relative standard curve method as outlined by the manufacturer (PE Applied Biosystems) and as described previously (17, 18). The mean glyceraldehyde-3-phosphate dehydrogenase concentration (primer set supplied by PE Applied Biosystems) was determined once for each cDNA sample and used to normalize expression of all other genes tested in the same sample. The relative difference in expression was recorded as the ratio of normalized target concentrations for the same cDNA dilution.

References

Hubank, M., and Schatz, D. G. (1994): Identifying differences in mRNA expression by representational difference analysis of cDNA. Nucleic Acids Res 22, 5640-8.

Phillips K. K., Welch D. R., Miele M. E., Lee J. H., Wei L. L., Weissman B. E. Suppression of MDA-MB-435 breast carcinoma cell metastasis following the introduction of human chromosome 11. Cancer Res., 56: 1222-1227, 1996.

Seraj M. J., Samant R. S., Verderame M. F., Welch D. R. Functional evidence for a novel human breast carcinoma metastasis suppressor, BRMS1, encoded at chromosome 11 q13. Cancer Res., 60: 2764-2769, 2000.

Uquidi, V., Sloan, D., Kawai, K., Agarwal, D., Woodman, A. C., Tarin, D. and Goodison, S. Contrasting expression of thrombospondin-1 and osteopontin correlates with absence or presence of metastatic phenotype in an isogenic model of spontaneous human breast cancer metastasis. Clin. Cancer Res., 8:61-74, 2002.

Although an exemplary embodiment of the invention has been described above by way of example only, it will be understood by those skilled in the field that modifications may be made to the disclosed embodiment without departing from the scope of the invention, which is defined by the appended claims.

1. A method for treatment of cancer comprising delivery of a non-metastatic agent to an individual.

2. The method as in claim 1 wherein the non-metastatic agent comprises a gene or gene product with increased expression in NM-2C5 cells as compared to M-4A4 cells.

3. The method as in claim 1 wherein the non-metastatic agent comprises a nucleic acid.

4. The method as in claim 3 wherein the nucleic acid comprises an expression cassette containing at least a portion of coding sequence for a gene selected from a group comprising matrix metalloproteinase-8, matrix metalloproteinase-17, tyrosinase related protein-1, cytokeratin-9 thrombospondin-1 and tissue inhibitor of matrix metalloproteinase-1.

5. The method as in claim 1 wherein the non-metastatic agent comprises a polypeptide.

6. The method as in claim 5 wherein the polypeptide comprises at least a portion of an amino acid sequence selected from a group comprising matrix metalloproteinase-8, matrix metalloproteinase-17, tyrosinase related protein-1, cytokeratin-9 thrombospondin-1 and tissue inhibitor of matrix metalloproteinase-1.

7. A method for treatment of cancer comprising the administration of a metastasis inhibiting agent.

8. The method as in claim 7 wherein the metastasis inhibiting agent comprises an agent that inhibits a gene or gene product with increased expression in M-4A4 cells as compared to NM-2C5 cells.

9. The method as in claim 7 wherein the metastasis inhibiting agent inhibits function of one from a group consisting of collagen IX, α-1, cytokeratin-9 and osteopontin.

10. A method for monitoring cancer comprising obtaining a sample from an individual and analyzing the sample for expression of a metastasis regulating gene or gene product.

11. The method as in claim 10 wherein expression of the metastasis regulating gene or gene product is different in the NM-2C5 and M-4A4 cell lines.

12. The method as in claim 10 wherein the metastasis regulating gene or gene product is selected from the group consisting of matrix metalloproteinase-8, matrix metalloproteinase-17, tyrosinase related protein-1, cytokeratin-9 thrombospondin-1, tissue inhibitor of matrix metalloproteinase-1, collagen IX, α-1, cytokeratin-9 and osteopontin.

13. The method as in claim 10 wherein the analysis is performed by a method selected from a group consisting of polymerase chain reaction, ELISA, northern, western and Southern blot, immunoassay and zymography.

14. The method as in claim 10 wherein the analysis is performed on a portion of a tumor.

15. The method as in claim 10 wherein the analysis is performed on a body fluid.

16. The method as in claim 10 wherein the analysis is performed to determine a prognosis for cancer.

17. The method as in claim 16 wherein the prognosis comprises a determination of a tendency of a patient to develop metastasis.

18. The method as in claim 10 wherein the analysis is performed to determine a diagnosis of cancer.
